(19) United States
(12) Patent Application Publication
(10) Pub. No.: US 2015/0018198 A1
(43) Pub. Date: Jan. 15, 2015

(54) METHOD FOR PREPARING CATALYSTS FOR PRODUCING ALCOHOLS FROM SYNTHESIS GAS

(71) Applicants: Arthur Jose Gerbasi da Silva, Rio de Janeiro (BR); Antonio Manzolillo Sanseverino, Rio de Janeiro (BR); Cristina Pontes Bittencourt Quitete, Rio de Janeiro (BR); Antonio Carlos Sallares de Mattos Carvalho, Petropolis (BR)

(72) Inventors: Arthur Jose Gerbasi da Silva, Rio de Janeiro (BR); Antonio Manzolillo Sanseverino, Rio de Janeiro (BR); Cristina Pontes Bittencourt Quitete, Rio de Janeiro (BR); Antonio Carlos Sallares de Mattos Carvalho, Petropolis (BR)

(73) Assignee: PETROLEO BRASILEIRO S.A. - PETROBRAS, Rio de Janeiro, RJ (BR)

(21) Appl. No.: 14/376,052

(22) PCT Filed: Jun. 13, 2012
(86) PCT No.: PCT/BR2012/000184

§ 371 Date: Aug. 25, 2014

(51) Int. Cl. B01J 27/051; C07C 29/153; B01J 27/232

(52) U.S. Cl. CPC B01J 27/0515; B01J 27/051; B01J 27/232; C07C 29/153
USPC 502/174; 502/220

(57) ABSTRACT

The present invention relates to a method of preparing catalysts based on molybdenum sulphide, with an alkaline promoter incorporated, said catalysts being employed in the production of alcohols, especially ethanol, from synthesis gas. The method involves reaction of molybdenum hexacarbonyl (Mo(CO)₆) with sulphur, so as to generate molybdenum sulphide, in which an alkaline promoter is then incorporated, so as to obtain a solid catalyst for application in processes of production of alcohols from synthesis gas, selective for ethanol.

### FIELD OF THE INVENTION

The present invention relates to the field of methods of preparing catalysts for producing alcohols, more particularly catalysts for producing ethanol and higher alcohols from synthesis gas. These catalysts comprise molybdenum sulphide, with an alkaline promoter incorporated, and allow processes of production of alcohols from synthesis gas to take place in less harsh operating conditions, especially with regard to the pressures employed.

### TECHNOLOGICAL BACKGROUND

The development of new technologies for producing fuels and synthetic chemicals using renewable sources, such as biomass, in place of fuels and chemicals of fossil origin, such as petroleum derivatives, has been pursued with the aim of combating climate change and improving energy security and air quality.

In this context, ethanol and the higher alcohols are regarded as an alternative for replacing gasoline in Otto cycle engines. Ethanol and the higher alcohols can also be used for the synthesis of various chemicals and polymers.

At present, ethanol is mainly produced by fermentation of sugars derived from biomass, especially sugars with 6 carbon atoms, whereas sugars with 5 carbon atoms and lignin, which are also present in biomass, are not used for producing ethanol. The higher alcohols are mainly produced from petroleum derivatives.

Gasification of biomass (or some other source of carbon and hydrogen), converting it to synthesis gas (mixture of carbon monoxide and hydrogen), followed by the catalytic conversion of this gas, could produce ethanol and higher alcohols in large quantities. However, catalytic conversion of synthesis gas to ethanol and higher alcohols faces various challenges and there is still no commercial process, even though research has already been conducted in this area for more than 90 years.

With the existing catalysts, synthesis of higher alcohols (mixture of alcohols with more than one carbon atom) from synthesis gas (mixture of carbon monoxide and hydrogen) is mainly carried out at high pressures (10.13 MPa to 15.20 MPa), in order to achieve adequate selectivity for the higher alcohols. This means large capital expenditure on equipment and a high cost of energy for compression of the synthesis gas.

Both homogeneous and heterogeneous catalytic processes have already been investigated.

The homogeneous catalytic processes for conversion of synthesis gas to ethanol are more selective, but require expensive catalysts, high pressures and complex methods for catalyst separation and recycling, making them uninteresting from a commercial standpoint.

The heterogeneous catalytic processes for conversion of synthesis gas to ethanol have low yields and low selectivity for ethanol, owing to the low initial rate of formation of the C—C bond and rapid reaction of the C2 intermediate formed (Subramani, V.; Gangwal, S. K. A Review of Recent Literature to Search for an Efficient Catalytic Process for the Conversion of Syngas to Ethanol. Energy & Fuels, v. 22, p. 814-839, 2008).

Recently there has been growing interest in the conversion of synthesis gas to ethanol and higher alcohols. However, there will need to be significant advances in catalyst design and in process development to make this conversion commercially attractive.

In 2008, Subramani and Gangwal (Subramani, V.; Gangwal, S. K. A Review of Recent Literature to Search for an Efficient Catalytic Process for the Conversion of Syngas to Ethanol. Energy & Fuels, v. 22, p. 814-839) undertook an extensive review of the catalytic routes for the conversion of synthesis gas to ethanol and higher alcohols.

The authors state that catalysts based on MoS₂ appear to be the most promising for converting synthesis gas to ethanol and higher alcohols, because they are more resistant to deactivation by sulphur and by coke deposits; they promote the formation of linear alcohols, with high selectivity for ethanol; and they are less sensitive to the presence of carbon dioxide in the synthesis gas. Still according to these authors, the conventional method of preparing catalysts based on MoS₂ is by the thermal decomposition or reduction of (NH₄)₂MoS₄.

Documents EP 0119609 A1, EP 0172431 A2 and U.S. Pat. No. 4,675,344 describe the preparation of sulphided catalysts, including those based on MoS₂, and refer to the methods of preparing catalysts described in the book Sulphide Catalysts, Their Properties and Applications, Otto Weisser and Stanislav Landa, pages 23 to 34, Pergamon Press, New York, 1973; and in U.S. Pat. No. 4,243,553 and U.S. Pat. No. 4,243,554.

Patent EP 0119609 A1 describes a process for producing alcohols from synthesis gas using a modified Fischer-Tropsch catalyst, which may or may not be sulphided, based on Mo and/or tungsten and/or rhenium, having a support and an alkaline promoter in addition to Co, Fe, or Ni.

Patent EP 0172431 A2 describes a process for producing alcohols from synthesis gas using a modified Fischer-Tropsch catalyst, which may or may not be sulphided, based on Mo and/or tungsten, with a support and an alkaline promoter in addition to Co, Fe, or Ni.

U.S. Pat. No. 4,675,344 describes a method for controlling the ratio of methanol to other alcohols obtained using a catalyst based on molybdenum and/or tungsten and adjustment of the flow of sulphur-containing compounds in the feed of process reactants.

However, in the literature there is neither description nor suggestion of a method of preparing catalysts for producing alcohols from synthesis gas, where the catalysts obtained display greater selectivity for ethanol, relative to the conventional catalysts, and the reaction of conversion of synthesis gas takes place at low pressures (5 MPa to 9 MPa).

### SUMMARY OF THE INVENTION

The present invention broadly relates to a method of preparing catalysts based on molybdenum sulphide, said catalysts being employed in the production of alcohols, especially ethanol, from synthesis gas.

The method comprises reaction of molybdenum hexacarbonyl (Mo(CO)₆) with sulphur)(S°, under inert atmosphere and employing an organic solvent, preferably p-xylene, capable of promoting the dissolution of sulphur in the reaction mixture, generating molybdenum sulphide, in which an alkaline promoter is then incorporated so as to obtain a solid catalyst for application in processes of production of alcohols from synthesis gas.

These catalysts, when employed in processes for producing higher alcohols from synthesis gas, display greater selectivity for ethanol than the known catalysts of the prior art, in addition to attaining a higher ethanol/methanol ratio, and allow these processes to operate at lower pressures (5 MPa to 9 MPa), i.e. in operating conditions that are less harsh, and therefore more economical.

### BRIEF DESCRIPTION OF THE DRAWINGS

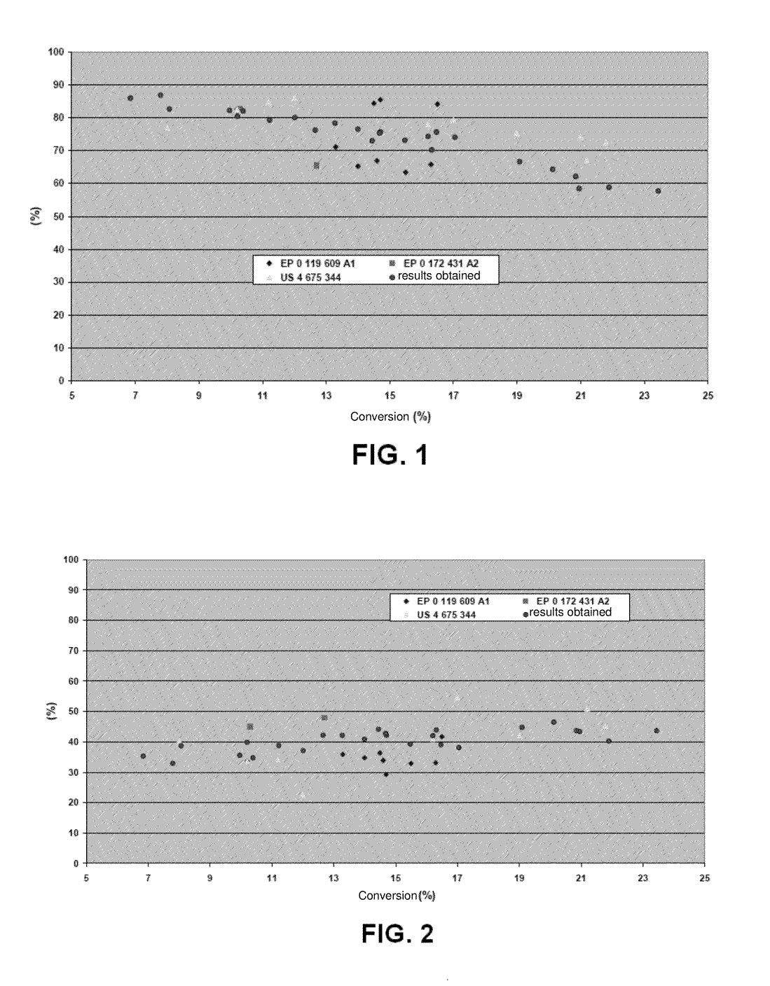

The appended FIG. 1 illustrates the relation between conversion and selectivity for total alcohols of catalysts for conversion of synthesis gas to ethanol and higher alcohols produced according to patents EP 0119609, EP 0172431 and U.S. Pat. No. 4,675,344 and a catalyst produced according to the present invention.

The appended FIG. 2 illustrates the relation between conversion and selectivity for higher alcohols of catalysts for conversion of synthesis gas to ethanol and higher alcohols produced according to patents EP 0119609, EP 0172431 and U.S. Pat. No. 4,675,344 and a catalyst produced according to the present invention.

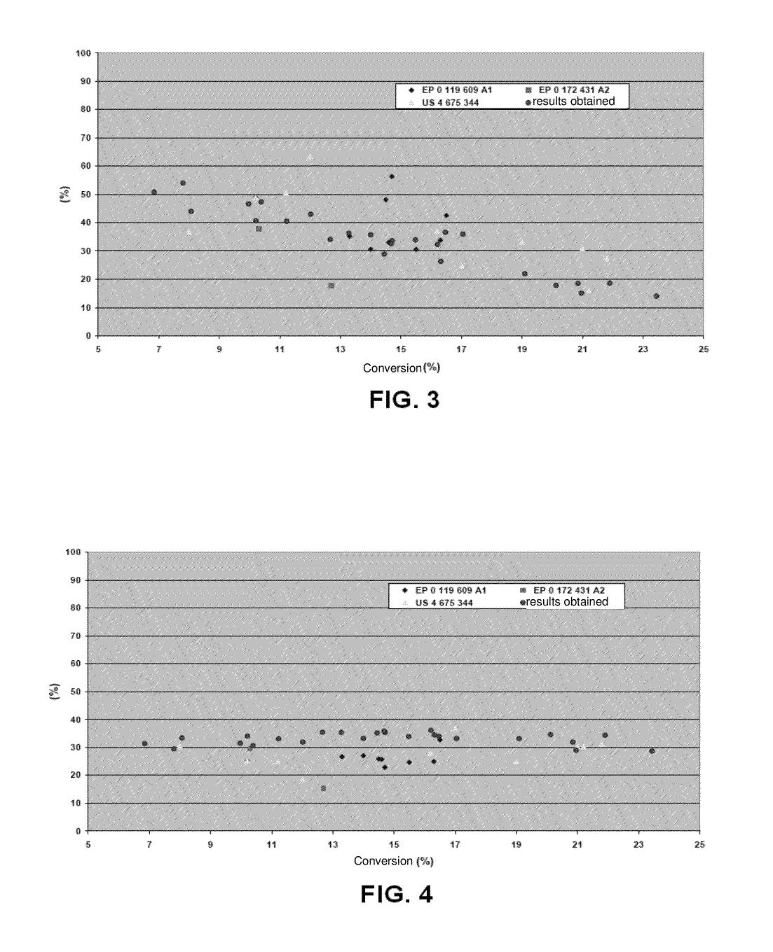

The appended FIG. 3 illustrates the relation between conversion and selectivity for methanol of catalysts for conversion of synthesis gas to ethanol and higher alcohols produced according to patents EP 0119609, EP 0172431 and U.S. Pat. No. 4,675,344 and a catalyst produced according to the present invention.

The appended FIG. 4 illustrates the relation between conversion and selectivity for ethanol of catalysts for conversion of synthesis gas to ethanol and higher alcohols produced according to patents EP 0119609, EP 0172431 and U.S. Pat. No. 4,675,344 and a catalyst produced according to the present invention.

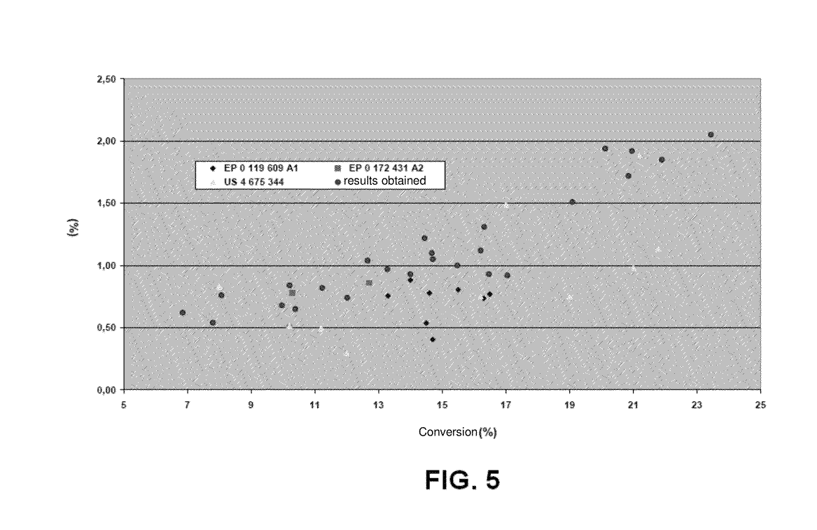

The appended FIG. 5 illustrates the relation between conversion and the ethanol/methanol selectivity ratio of catalysts for conversion of synthesis gas to ethanol and higher alcohols produced according to patents EP 0119609, EP 0172431 and U.S. Pat. No. 4,675,344 and a catalyst produced according to the present invention.

### DETAILED DESCRIPTION OF THE INVENTION

The present invention relates to a method of preparing catalysts for producing alcohols, especially ethanol, from synthesis gas (mixture of carbon monoxide and hydrogen), with high selectivity with respect to ethanol, compared to conventional catalysts.

The method relates broadly to the preparation of a catalyst based on molybdenum sulphide generated by the reaction of molybdenum hexacarbonyl with sulphur, under inert atmosphere, employing an organic solvent, preferably p-xylene, for promoting the conversion of synthesis gas (CO+H₂) to alcohols, especially ethanol. In this case, the organic solvent does not participate effectively in the reaction, but by promoting the dissolution of sulphur it facilitates the reactions of conversion, on account of greater interaction between reactants.

The method of preparing catalysts according to the present invention comprises the following steps:
a) adding an organic solvent to a reaction vessel, this then being filled with an inert gas so that in the reaction vessel the proportion of solvent is 1/3 and that of inert gas is 2/3 (by volume);
b) adding sulphur, under inert atmosphere and with reflux, to the reaction vessel containing the mixture of solvent and inert gas, so that the sulphur/solvent ratio is 0.0145 (by weight);
c) heating the mixture obtained in (b) to temperatures between 20° C. and 140° C., for a period of time between 5 and 20 minutes, preferably for 10 minutes, until all the sulphur has dissolved, and then cooling the mixture to room temperature (between 20° C. and 30° C.);
d) adding molybdenum hexacarbonyl (Mo(CO)₆) to the mixture, so that the S/Mo(CO)₆ ratio is 0.242 (by weight);
e) heating the mixture obtained in (d) to 140° C., maintaining this temperature for a period of time from 5 to 180 minutes, preferably 150 minutes, until there is formation of a black powder comprising molybdenum sulphide;
f) dry filtering of the black powder of molybdenum sulphide formed with the aid of a drying agent, then submitting the filtrate to a thermal treatment, under a stream of inert gas, for a period of time from 30 to 120 minutes, preferably 60 minutes, at a temperature varying from 500° C. to 700° C., preferably 550° C.;
g) adding an alkaline promoter to the black powder of molybdenum sulphide, already filtered and submitted to thermal treatment, and triturating the resultant mixture until it is homogeneous, the atomic ratio between alkaline promoter and Mo being between 0.1 and 1.0;
h) drying the product obtained from the mixture of molybdenum sulphide and alkaline promoter, under a stream of inert gas.

In this method it is important to maintain an inert atmosphere throughout catalyst preparation, since oxygen, if present in the reaction mixture, exerts an oxidizing action on the molybdenum sulphide formed, thus altering its catalytic activity.

Among the inert gases useful for the present invention, we may mention: argon, nitrogen and helium, among others.

To keep the reaction mixture free from oxygen, it is also important to use an organic solvent that has been degassed, or preferably is oxygen-free.

As well as being oxygen-free, said solvent must also display other characteristics, such as promoting complete dissolution of the reactants, and have a boiling point between 130° C. and 145° C.

Among the organic solvents useful for the present invention, we may mention: m-xylene, o-xylene, p-xylene, or a mixture thereof in any proportions.

As p-xylene has a boiling point close to 140° C. and good capacity for dissolution of the reactants, it is the preferred solvent.

Another advantage of p-xylene is that it can be degassed by cooling liquid p-xylene until it solidifies, followed by heating under vacuum, until it returns to the liquid phase. Removal of oxygen (degasification) is easier when p-xylene is used, as it has a crystallization temperature of 13° C.

To promote the reaction of molybdenum hexacarbonyl with sulphur, it is recommended to heat the reaction mixture at temperatures in the range from 50° C. to 140° C., preferably temperatures close to the boiling point of the organic solvent employed for dissolving the sulphur, more preferably 140° C., a temperature that is close to the boiling point of p-xylene, which is 138.5° C.

The product of the reaction of molybdenum hexacarbonyl with sulphur basically comprises molybdenum disulphide (MoS₂), and the reaction mixture may also contain other types of molybdenum sulphide, such as: Mo₃S₄, and Mo₂S₃, among others.

The molybdenum sulphide is separated from the reaction mixture by filtration of the molybdenum sulphide, with the aid of a drying agent, which may be, among others: ketones, alcohols comprising 1 to 3 carbon atoms, ethyl acetate, toluene and carbon tetrachloride.

Among the alcohols, we may mention: methanol, ethanol, propanol and isopropanol, more preferably ethanol, as it has low cost and low toxicity, as well as being less harmful to the environment.

Moreover, among the ketones, preferably acetone is used, for the same reasons as already mentioned for ethanol.

After filtration of the molybdenum sulphide, it undergoes a thermal treatment, promoted by raising the temperature to the desired range, which is between 500° C. and 700° C., the temperature being increased slowly at 1° C./min, so as to induce crystallization of the particles of MoS₂.

For producing catalysts for conversion of synthesis gas to alcohols, especially ethanol, it is necessary to incorporate alkaline promoters, since the catalysts based only on molybdenum sulphide, without the presence of a promoter, if used in this type of reaction, would generate light hydrocarbons as main products.

Among the alkaline promoters useful for the method of the present invention we have Cs₂CO₃, Rb₂CO₃, preferably, K₂CO₃.

Moreover, with the same methods of catalyst preparation as described above, in the step of adding the promoter, this can also be added by incipient wet impregnation as opposed to physical mixing. In this case the alkaline promoter is mixed with the resultant black powder of molybdenum sulphide in a roller mixer, or some other type of mixer, for approximately 2 hours.

Besides having alkaline promoters incorporated, the catalyst may also have transition metals incorporated such as Ni, Co or Rh, in proportions from 0.1% to 0.5% relative to the weight of catalyst.

Transition metals are additives, or co-catalysts, that may improve catalyst performance. In the case of Ni and Co, these help in the reaction of homologation of methanol (transformation of methanol to ethanol).

The catalyst, based on molybdenum sulphide, of the present invention is produced in powder form and may be used for producing “pellets”, which are then used in reactors that form part of the process equipment used for conversion of synthesis gas to alcohols.

The catalysts produced according to the method of preparation of the present invention have density from 1.2 g/cm³ to 3 g/cm³, average pore size from 10 nm to 13 nm, total pore volume from 0.01 m³/g to 0.06 m³/g and BET surface area from 5 m²/g to 21 m²/g.

Therefore, as shown in the examples, the method of preparing catalysts of the present invention allows the production of catalysts for use in processes of conversion of synthesis gas to alcohols, especially ethanol, at low pressures (5 MPa to 9 MPa), where said catalysts comprise molybdenum sulphide with an alkaline promoter incorporated.

The following examples illustrate the method of preparing catalysts based on molybdenum sulphide with an alkaline promoter incorporated and application thereof in processes of conversion of synthesis gas to ethanol and higher alcohols, without the scope of the invention being limited thereby.

### Example 1

This example illustrates the method of preparing a catalyst for processes of conversion of synthesis gas to ethanol and higher alcohols according to the present invention.

A vessel containing 100 ml of p-xylene is cooled in liquid nitrogen until the p-xylene solidifies. Next, the product is subjected to vacuum and is then heated until it returns to the liquid phase. This procedure is repeated twice and finally the vessel is filled with nitrogen.

An amount of approximately 1.25 g of sulphur is then added to the vessel containing p-xylene, under nitrogen atmosphere, with connection of a nitrogen supply and a reflux column.

The temperature of the mixture is increased until it reaches 140° C. for an interval of time of 30 minutes and is maintained at this value until all the sulphur has dissolved (approximately 10 minutes). Then the mixture is cooled to room temperature.

An amount of approximately 5.15 g of molybdenum hexacarbonyl, Mo(CO)₆ is added and the temperature is increased to 140° C. in 20 minutes. After 150 minutes at this temperature, the mixture obtained from the reaction is cooled to room temperature.

The black powder obtained is then filtered and dried with the aid of acetone, and is then submitted to a thermal treatment in a tubular furnace at a temperature of 550° C. for one hour, reached with application of a heating ramp of 1° C./min, supplied with a nitrogen stream with a flow rate of 100 ml/min.

K₂CO₃ is triturated together with the powder resulting from the reaction in such a way that the physical mixture obtained from the two powders is homogeneous and has an atomic ratio of K to Mo equivalent to 0.7.

Finally, the catalyst undergoes drying in a tubular furnace at a temperature of 110° C., reached with application of a heating ramp of 2° C./min, with a nitrogen stream of 100 ml/min for 16 hours.

### Example 2

This example illustrates tests for producing higher alcohols from synthesis gas using catalysts prepared as described in the present invention, where a stream of synthesis gas with H₂/CO ratio of between 1.0 and 2.0 and a content of H₂S between 50 ppm and 100 ppm comes into contact with a catalyst bed at a temperature in the range from 260° C. to 340° C., a pressure of 50 bar and GHSV between 1000 and 5000 h⁻¹.

The results obtained for a period of time of 200 hours for each test are shown in Tables 1 and 2 below.

Table 1 gives the results achieved in terms of productivity, or percentage mass flow rates of CO that are converted to higher alcohols (in this case, alcohols containing from 2 to 4 carbon atoms), ethanol and methanol.

TABLE 1

| | Productivity of | | |
|---|---|---|---|
| Conversion of | higher alcohols | Productivity of | Productivity of |
| CO (%) | (%) | ethanol (%) | methanol (%) |
| 0-25 | 0-7.3 | 0-5.1 | 0-4.6 |

Table 2 below presents the results, in terms of selectivity for ethanol, methanol and ratio of ethanol and methanol selectivities, as well as the operating conditions applied in the tests (pressure, GHSV and temperature).

TABLE 2

| | Selectivity | | | | Selectivity | | | |
|---|---|---|---|---|---|---|---|---|
| | for | | | | for | Selectivity | Selectivity | |
| | Total | | | Ratio of | Higher | for | for | |
| | Alcohols | | | EtOH/MeOH | Alcohols | Ethanol | Methanol | |
| | (%) | | | selectivities | (%) | (%) | (%) | |
| Conversion | without | Pressure | GHSV | without | without | without | without | Temp |
| (%) | CO₂ | (MPa) | (h⁻¹) | CO₂ | CO₂ | CO₂ | CO₂ | (° C.) |
| 23.44 | 57.68 | 5.0 | 1612.50 | 2.05 | 43.68 | 28.68 | 14.00 | 320 |
| 20.12 | 64.30 | 5.0 | 1535.49 | 1.94 | 46.48 | 34.53 | 17.82 | 300 |
| 14.71 | 75.73 | 5.0 | 2457.44 | 1.05 | 42.13 | 35.34 | 33.60 | 300 |
| 16.47 | 75.60 | 5.0 | 3194.67 | 0.93 | 39.04 | 33.88 | 36.56 | 300 |
| 16.32 | 70.22 | 5.0 | 2687.10 | 1.31 | 43.93 | 34.44 | 26.29 | 300 |
| 13.28 | 78.33 | 5.0 | 2777.97 | 0.97 | 42.07 | 35.30 | 36.26 | 300 |
| 14.68 | 75.29 | 5.0 | 2457.44 | 1.10 | 42.76 | 35.81 | 32.53 | 300 |
| 15.48 | 73.13 | 5.0 | 3194.67 | 1.00 | 39.24 | 33.83 | 33.89 | 300 |
| 14.00 | 76.54 | 5.0 | 9106.29 | 0.93 | 40.82 | 33.20 | 35.72 | 320 |
| 10.21 | 80.49 | 5.0 | 3993.33 | 0.84 | 39.82 | 33.99 | 40.67 | 300 |
| 14.45 | 73.03 | 5.0 | 4903.39 | 1.22 | 44.14 | 35.17 | 28.89 | 320 |
| 6.85 | 85.99 | 5.0 | 9806.77 | 0.62 | 35.23 | 31.32 | 50.76 | 300 |
| 9.97 | 82.21 | 5.0 | 5324.44 | 0.68 | 35.59 | 31.47 | 46.62 | 300 |
| 7.80 | 86.89 | 5.0 | 6709.90 | 0.54 | 32.89 | 29.37 | 54.00 | 300 |
| 10.39 | 82.04 | 5.0 | 3194.67 | 0.65 | 34.74 | 30.61 | 47.30 | 300 |
| 12.66 | 76.20 | 5.0 | 5312.00 | 1.04 | 42.15 | 35.42 | 34.05 | 320 |
| 8.07 | 82.65 | 5.0 | 8499.20 | 0.76 | 38.67 | 33.34 | 43.98 | 320 |
| 12.02 | 80.03 | 5.0 | 3194.67 | 0.74 | 37.09 | 31.87 | 42.94 | 300 |
| 19.09 | 66.64 | 5.0 | 2777.97 | 1.51 | 44.73 | 33.11 | 21.91 | 320 |
| 20.96 | 58.42 | 5.0 | 1791.40 | 1.92 | 43.38 | 28.91 | 15.04 | 320 |
| 17.05 | 74.04 | 5.0 | 3194.60 | 0.92 | 38.11 | 33.14 | 35.93 | 300 |
| 20.85 | 62.14 | 5.0 | 2457.44 | 1.72 | 43.63 | 31.86 | 18.51 | 320 |
| 21.90 | 58.81 | 5.0 | 1612.26 | 1.85 | 40.20 | 34.35 | 18.61 | 300 |
| 16.21 | 74.32 | 5.0 | 4563.81 | 1.12 | 41.99 | 36.07 | 32.33 | 300 |
| 11.23 | 79.32 | 5.0 | 3549.63 | 0.82 | 38.8 | 33.03 | 40.52 | 300 |

### Example 3

This example illustrates the textural properties of catalysts produced according to the method of the present invention.

Table 3 below illustrates the textural properties (average pore size, total pore volume and surface area) of catalysts produced according to the present invention.

The catalysts described in Table 3 below were prepared according to the method of the present invention, incorporation of alkaline promoter (K, Cs or Rb) having been carried out by physical mixing (identified as MF in the table) or wet impregnation (identified in the table as VU).

Moreover, for the catalysts in Table 3 below, their atomic ratios of alkaline promoter relative to molybdenum are shown, together with the percentage by weight of transition metal relative to the total weight of catalyst. In this case, the catalyst “0.1% Rh-0.3Rb/VU” in Table 3 refers to a catalyst with a percentage by weight of 0.1% of Rh, impregnated by the wet process, with an atomic ratio of 0.3 of Rb/Mo.

TABLE 3

| | Average | Total pore | |
|---|---|---|---|
| | pore size | volume | Surface area¹ |
| CATALYST | (nm) | (m³/g) | (m²/g) |
| 0.7K/MF | 11.7 | 0.057 | 20.7 ± 0.1 |
| 0.3Cs/MF | 12.2 | 0.029 | 9.36 ± 0.04 |
| 0.3Rb/MF | 12.0 | 0.034 | 11.2 ± 0.02 |
| 0.7Rb/MF | 12.5 | 0.017 | 5.31 ± 0.02 |
| 0.33Ni—0.3Rb/MF | 12.4 | 0.028 | 9.12 ± 0.02 |
| 0.1% Rh—0.3Rb/VU | 10.9 | 0.026 | 9.51 ± 0.02 |
| 0.5% Rh—0.3Rb/VU | 12.1 | 0.042 | 13.8 ± 0.02 |
| 0.1% Co—0.3Rb/VU | 11.0 | 0.041 | 14.9 ± 0.05 |
| 0.5% Co—0.3Rb/VU | 11.1 | 0.042 | 15.2 ± 0.02 |
| 0.5% Ni—0.3Rb/VU | 11.4 | 0.042 | 14.8 ± 0.02 |
| 0.25% Rh—0.25% Co—0.3Rb/ | 10.7 | 0.047 | 17.6 ± 0.02 |
| VU | | | |
| 0.25% Co—0.25% Ni—0.3Rb/ | 10.8 | 0.042 | 15.6 ± 0.02 |
| VU | | | |
| 0.167% Rh—0.167% Co—0.167% | 10.9 | 0.039 | 14.5 ± 0.02 |
| Ni—0.3Rb/VU | | | |
| 0.1% Rh—0.3K/US-PM | 11.3 | 0.059 | 20.8 ± 0.04 |
| 1% Rh—0.3K/US-PM | 11.3 | 0.052 | 18.5 ± 0.02 |
| 0.5% Co—0.3K/US-PM | 11.4 | 0.052 | 18.3 ± 0.03 |
| 0.25% Co—0.25% Ni—0.3K/ | 11.8 | 0.052 | 17.7 ± 0.03 |
| PM | | | |
| 0.167% Rh—0.167% Co—0.167% | 12.8 | 0.058 | 17.9 ± 0.02 |
| Ni—0.3K/PM | | | |

Note
¹Surface area calculated by the BET method.

### Comparative Example 1

This example illustrates the performance, with respect to selectivity, of catalysts of the prior art when employed in a process for conversion of synthesis gas to ethanol and higher alcohols.

The results obtained, in terms of selectivity, using catalysts described in patent documents EP 0119609, EP 0172431 and U.S. Pat. No. 4,675,344 in processes of conversion of synthesis gas to ethanol and higher alcohols, are presented in Tables 4 and 5 below and relate to processes employing sulphided catalysts based on molybdenum assuming a range of conversion of between 5% and 25%.

TABLE 4

| | | Selectivity | | | Ratio of |
|---|---|---|---|---|---|
| | | for Total | | | EtOH/MeOH |
| | | Alcohols | | | selectivities |
| | Conversion | (%) without | Pressure | GHSV | without |
| PATENT | (%) | CO₂ | (bar) | (h⁻¹) | CO₂ |
| EP 0 119 609 | 10.2-16.5 | 63.4-85.53 | 79.9-207.5 | 676-3900 | 0.40-0.88 |
| A1 | | | | | |
| EP 0 172 431 | 10.3-12.7 | 65.5-82.8 | 104.4 | 614-2200 | 0.78-0.86 |
| A2 | | | | | |
| U.S. Pat. | 8-21.8 | 67.05-85.99 | 103.0-208.5 | 1980-5220 | 0.29-1.88 |
| No. 4,675,344 | | | | | |

TABLE 5

| | | | | | | | Selectivity | Selectivity | |
|---|---|---|---|---|---|---|---|---|---|
| | | | | | | | for | for | Ratio of |
| | | | | | S1 | S2 | Ethanol | Methanol | EtOH/MeOH |
| | | | | | (%) | (%) | (%) | (%) | selectivities |
| | C¹ | T² | P³ | GHSV⁴ | without | without | without | without | (%) without |
| Patents | (%) | ° C. | (bar) | (h−1) | CO₂ | CO₂ | CO₂ | CO₂ | CO₂ |
| EP | 14.7 | 260 | 81.6 | 1283 | 85.53 | 29.23 | 22.8 | 56.3 | 0.40 |
| 0 119 609 | | | | | | | | | |
| A1 | | | | | | | | | |
| EP | 16.5 | 262 | 79.9 | 676 | 84.2 | 41.7 | 32.7 | 42.5 | 0.77 |
| 0 119 609 | | | | | | | | | |
| A1 | | | | | | | | | |
| EP | 16.3 | 255 | 102.0 | 3171 | 65.8 | 33.1 | 24.9 | 33.8 | 0.74 |
| 0 119 609 | | | | | | | | | |
| A1 | | | | | | | | | |
| EP | 13.3 | 255 | 91.8 | 2254 | 71.1 | 35.9 | 26.6 | 35.2 | 0.76 |
| 0 119 609 | | | | | | | | | |
| A1 | | | | | | | | | |
| EP | 14.6 | 258 | 91.8 | 3140 | 66.9 | 33.9 | 25.7 | 33 | 0.78 |
| 0 119 609 | | | | | | | | | |
| A1 | | | | | | | | | |
| EP | 15.5 | 250 | 91.8 | 2300 | 63.4 | 32.9 | 24.6 | 30.5 | 0.81 |
| 0 119 609 | | | | | | | | | |
| A1 | | | | | | | | | |
| EP | 14 | 250 | 91.8 | 1934 | 65.3 | 34.7 | 27.0 | 30.6 | 0.88 |
| 0 119 609 | | | | | | | | | |
| A1 | | | | | | | | | |
| EP | 10.2 | 260 | 136.1 | 3150 | 82.7 | 33.7 | 25.2 | 49 | 0.51 |
| 0 119 609 | | | | | | | | | |
| A1 | | | | | | | | | |
| EP | 14.5 | 265 | 207.5 | 3900 | 84.4 | 36.3 | 25.8 | 48.1 | 0.54 |
| 0 119 609 | | | | | | | | | |
| A1 | | | | | | | | | |
| EP | 10.3 | 295 | 104.4 | 2200 | 82.8 | 45 | 29.5 | 37.8 | 0.78 |
| 0 172 431 | | | | | | | | | |
| A2 | | | | | | | | | |
| EP | 12.7 | 350 | 104.4 | 614 | 65.5 | 47.8 | 15.2 | 17.7 | 0.86 |
| 0 172 431 | | | | | | | | | |
| A2 | | | | | | | | | |
| U.S. Pat. No. | 8 | 268 | 103.0 | 1980 | 77 | 40.33 | 30.39 | 36.67 | 0.83 |
| 4,675,344 | | | | | | | | | |
| U.S. Pat. No. | 12 | 270 | 137.1 | 3000 | 85.99 | 22.52 | 18.66 | 63.47 | 0.29 |
| 4,675,344 | | | | | | | | | |
| U.S. Pat. No. | 19 | 312 | 137.1 | 4200 | 75.25 | 42.15 | 24.8 | 33.1 | 0.75 |
| 4,675,344 | | | | | | | | | |
| U.S. Pat. No. | 21.2 | 320 | 171.1 | 3348 | 67.05 | 50.95 | 30.3 | 16.1 | 1.88 |
| 4,675,344 | | | | | | | | | |
| U.S. Pat. No. | 17 | 302 | 205.1 | 2310 | 79.3 | 54.6 | 36.8 | 24.7 | 1.49 |
| 4,675,344 | | | | | | | | | |
| U.S. Pat. No. | 10.2 | 296 | 137.1 | 3150 | 82.5 | 33.6 | 25.2 | 48.9 | 0.52 |
| 4,675,344 | | | | | | | | | |
| U.S. Pat. No. | 21 | 260 | 164.3 | 3075 | 74.1 | 43.4 | 30.1 | 30.7 | 0.98 |
| 4,675,344 | | | | | | | | | |
| U.S. Pat. No. | 21.8 | 275 | 157.5 | 3195 | 72.5 | 45.1 | 31.1 | 27.4 | 1.14 |
| 4,675,344 | | | | | | | | | |
| U.S. Pat. No. | 16.2 | 282 | 174.5 | 3390 | 77.9 | 40.9 | 27.8 | 37 | 0.75 |
| 4,675,344 | | | | | | | | | |
| U.S. Pat. No. | 11.2 | 275 | 208.5 | 5220 | 84.7 | 34.2 | 25.0 | 50.5 | 0.50 |
| 4,675,344 | | | | | | | | | |

Notes:
¹C = Conversion;
²T = Temperature;
³P = Pressure;
⁴GHSV = “Gas Hourly Space Velocity”;
5—S1 = Selectivity for Total Alcohols;
6—S2 = Selectivity for Higher alcohols.

The results obtained and presented in Table 5 above were plotted in figures (graphs) of the relation of conversion and selectivity; these figures are an integral part of the present invention.

From analysis of the results and figures, or graphs, illustrating the present invention, it can be seen that the productivity and the selectivity for total alcohols and higher alcohols of the catalysts produced according to the present invention are comparable to the results presented in patent documents EP 0119609, EP 0172431 and U.S. Pat. No. 4,675,344, included in the prior art.

It should be pointed out that in terms of selectivity for ethanol, FIG. 4, better performance is found for the catalyst obtained according to the method of the present invention, and moreover, in general, it has higher values for the ratios of ethanol and methanol selectivities, FIG. 5, than the catalysts produced according to the patent documents cited above.

1. METHOD OF PREPARING CATALYSTS FOR PRODUCING ALCOHOLS FROM SYNTHESIS GAS, characterised in that it comprises the following steps:
a) adding an organic solvent to a reaction vessel, this then being filled with an inert gas so that in the reaction vessel the proportion of solvent is 1/3 and of inert gas is 2/3 (by volume);
b) adding sulphur, under inert atmosphere and with reflux, to the reaction vessel containing the mixture of solvent and inert gas, so that the sulphur/solvent ratio is 0.0145 (by weight);
c) heating the mixture obtained in (b) to temperatures between 20° C. and 140° C., for a period of time between 5 and 20 minutes, until all the sulphur has dissolved, and then cooling the mixture to room temperature (between 20° C. and 30° C.);
d) adding molybdenum hexacarbonyl (Mo(CO)₆) to the mixture, so that the S/Mo(CO)₆ ratio is 0.242 (by weight);
e) heating the mixture obtained in (d) to 140° C., maintaining this temperature for a period of time from 5 to 180 minutes, until there is formation of a black powder comprising molybdenum sulphide;
f) dry filtration of the black powder of molybdenum sulphide formed with the aid of a drying agent, the filtrate then being submitted to thermal treatment, under a stream of inert gas, for a period of time from 30 to 120 minutes, at a temperature varying from 500° C. to 700° C.;
g) adding an alkaline promoter to the black powder of molybdenum sulphide, already filtered and submitted to thermal treatment, and triturating the resultant mixture until homogeneous, the atomic ratio of alkaline promoter to Mo being in a range between 0.1 and 1.0;
h) drying the product obtained from the mixture of molybdenum sulphide and alkaline promoter, under a stream of inert gas, at a temperature of 110° C.

2. METHOD according to claim 1, characterised in that the inert gas is selected from: argon, nitrogen and helium.

3. METHOD according to claim 1, characterised in that the organic solvent is oxygen-free and has a boiling point between 130° C. and 145° C.

4. METHOD according to claim 1, characterised in that the organic solvent is selected from: m-xylene, o-xylene, p-xylene, or a mixture thereof in any proportions.

5. METHOD according to claim 1, characterised in that the time required for dissolution of sulphur in the organic solvent (step c) is preferably 10 minutes.

6. METHOD according to claim 1, characterised in that the time required for formation of the black powder of molybdenum sulphide (step e) is preferably 150 minutes.

7. METHOD according to claim 1, characterised in that the drying agent is selected from: ketones, alcohols comprising 1 to 3 carbon atoms, ethyl acetate, toluene and carbon tetrachloride.

8. METHOD according to claim 7, characterised in that the ketone is preferably acetone.

9. METHOD according to claim 7, characterised in that the alcohol is selected from: methanol, ethanol, propanol and isopropanol.

10. METHOD according to claim 1, characterised in that the alkaline promoter is selected from: Cs₂CO₃, Rb₂CO₃ and K₂CO₃.

11. METHOD according to claim 1, characterised in that, alternatively, the alkaline promoter is incorporated in the molybdenum sulphide by incipient wet impregnation.

12. METHOD according to claim 1, characterised in that it contains an additional step of incorporation of transition metals in proportions from 0.1% to 0.5% relative to the weight of catalyst.

13. METHOD according to claim 12, characterised in that the transition metal is selected from: Ni, Co or Rh.

14. CATALYSTS FOR PRODUCING ETHANOL AND HIGHER ALCOHOLS, prepared according to the method described in claim 1, characterised in that they have density from 1.2 g/cm³ to 3 g/cm³, average pore size from 10 nm to 13 nm, total pore volume from 0.01 m³/g to 0.06 m³/g and BET surface area from 5 m²/g to 21 m²/g.
